(19) United States
(12) Patent Application Publication
(10) Pub. No.: US 2025/0144260 A1
(43) Pub. Date: May 8, 2025

(54) STERILIZATION ROBOT

(71) Applicant: LG ELECTRONICS INC., Seoul (KR)

(72) Inventors: Sumi KIM, Seoul (KR); Jonghun KWON, Seoul (KR); Junhee HAN, Seoul (KR); Hyunju OH, Seoul (KR); Eunki KIM, Seoul (KR)

(73) Assignee: LG ELECTRONICS INC., Seoul (KR)

(21) Appl. No.: 18/838,169

(22) PCT Filed: Feb. 14, 2022
(86) PCT No.: PCT/KR2022/002121

§ 371 Date: Aug. 13, 2024

(51) Int. Cl. A61L 2/24; A61L 2/10

(52) U.S. Cl. CPC A61L 2/24; A61L 2/10; A61L 2202/14; A61L 2202/16

(57) ABSTRACT

A sterilization robot comprises: a case; a traveling unit, which is positioned beneath the case and provides a traveling function; a sterilization module positioned on a side surface of the case; a sensing unit for sensing an adjacent object; and a control unit for setting a traveling path on the basis of information about the object sensed by the sensing unit, and controlling the traveling unit so that the sterilization robot moves along the traveling path, and controls the traveling unit and the sterilization module with an optimal scheme such that sterilization efficiency can be increased.

### TECHNICAL FIELD

The disclosure relates to a sterilization robot that sterilizes objects, while moving freely.

### BACKGROUND ART

To take charge of a portion of factory automation, robots have been developed for industrial use. Recently, the application range of robots has been further expanded, and robots that may be used in daily life as well as medical robots and aerospace robots are being developed.

Due to the recent epidemic, the need for regular sterilization is increasing for epidemic prevention. In particular, hospital rooms, operating rooms, and other spaces where many people move around in hospitals should be sterilized frequently. Public spaces with a large number of people and rooms such as hotels should also be sanitized after each change of guests.

Although chemical disinfection is also performed, the use of ultraviolet (UV) light for sterilization has the advantage of high sterilization efficiency with minimal impact on the human body. However, since the disinfection work should be repeated periodically, cost increase caused by the input of manpower is a problem, and the need for robots with sterilization functions has recently increased.

In some cases, a sterilization function is added to a robot vacuum cleaner. However, this type of robot has the limitation of not being able to sterilize areas where human hands and droplets may reach, and thus a robot capable of sterilizing above it is needed.

### DISCLOSURE

### Technical Problem

An aspect of the disclosure is to provide a sterilization robot for increasing sterilization efficiency by performing sterilization in an optimal manner.

Another aspect of the disclosure is to provide a sterilization robot for minimizing discoloration caused by ultraviolet irradiation by avoiding sterilization of unnecessary parts and redundant sterilization.

Another aspect of the disclosure is to provide a sterilization robot for determining whether sterilization is necessary and, if sterilization is not necessary, increasing its traveling speed to quickly complete sterilization.

### Technical Solution

Provided is a sterilization robot including: a case; a traveling unit located in a lower part of the case and providing a traveling function; a sterilization module located on a side surface of the case; a sensing unit detecting a nearby object; and a controller setting a traveling path based on object information detected by the sensing unit and controlling the traveling unit to move along the traveling path.

The controller may determine a sterilization object among objects detected by the sensing unit, and set the traveling path to pass by the sterilization object while maintaining a sterilization distance from the sterilization object.

When a movement-direction width of the sterilization robot is equal to or greater than a first threshold width, the controller may set a first travel path to maintain a first distance from the sterilization object and operate the sterilization robot in a first mode.

A pair of sterilization modules may be located on both sides in a direction perpendicular to a traveling direction of the traveling unit, and the controller may turn on a sterilization module facing the sterilization object and turn off a sterilization module located opposite thereto, in the first mode.

When there is no sterilization object among the detected objects, the controller may set a second traveling path for movement along a wall and operate the sterilization robot in a second mode.

The traveling unit may move at a first speed in the first mode and at a second speed in the second mode.

The controller may turn off the sterilization modules in the second mode.

When the movement-direction width of the sterilization robot has a value between a second threshold width and the first threshold width, the controller may set a third traveling path running along a middle of the movement-direction width and operate the sterilization robot in a third mode.

A pair of sterilization modules may be located on both sides in a direction perpendicular to a traveling direction of the traveling unit, and the controller may turn on the pair of sterilization modules in the third mode and control the traveling unit to move at a third speed.

When the third traveling path ends at a dead end, the controller may control the traveling unit to reverse a traveling direction turn around, and move back along the third traveling path.

When the sterilization object is a door, the first traveling path may be set to be spaced from the door by a second distance greater than the first distance.

When the set traveling path overlaps with a previously traveled traveling path, the controller may control the traveling unit to move at a speed greater than a first speed used to move along the previously traveled traveling path.

The controller may store a traveling starting point by moving to an object at a location closest to a departure point, and upon arrival at the traveling starting point, turn off the traveling unit and the sterilization module.

When an unsterilized area remains upon arrival at the traveling starting point, the controller may control the traveling unit and the sterilization module by setting a traveling path that does not overlap with a previously traveled traveling path.

The sterilization module may include: a sterilization lamp emitting ultraviolet light; and a reflector reflecting the ultraviolet light on a rear surface of the sterilization lamp.

The sterilization module may include an actuator protruding from the case or changing an angle.

The actuator may include a plurality of actuators on a rear surface of the sterilization module, and the controller may control a position and a direction of the sterilization module by controlling a length change of the actuators.

When the sterilization object is highly contaminated, the controller may change the direction of the sterilization module during traveling of the sterilization robot by controlling the actuator to direct the sterilization module toward the sterilization object.

### Advantageous Effects

A sterilization robot according to at least one embodiment of the disclosure may increase sterilization efficiency by controlling a traveling unit and a sterilization module in an optimal manner.

Further, a sterilization robot according to at least one embodiment of the disclosure may minimize discoloration caused by ultraviolet irradiation by avoiding sterilization of unnecessary parts and redundant sterilization.

Further, a sterilization robot according to at least one embodiment of the disclosure may determine whether sterilization is necessary and, if sterilization is not necessary, increase its traveling speed to quickly complete sterilization.

Further, the sterilization robot may include a surface without a sterilization lamp, such that a user may access and operate the sterilization robot to cope with an emergency.

Further, the sterilization robot may efficiently irradiate ultraviolet light toward a sterilization object by adjusting the position and angle of the sterilization module.

The sterilization robot may set an accurate traveling path using a lidar that detects movement in various directions and a sensor such as a proximity sensor.

It will be appreciated by persons skilled in the art that the effects that may be achieved with the present disclosure are not limited to what has been particularly described hereinabove and other advantages of the present disclosure will be more clearly understood from the following detailed description taken in conjunction with the accompanying drawings.

### BRIEF DESCRIPTION OF THE DRAWINGS

FIG. 1 is a block diagram illustrating the configuration of a sterilization robot according to an embodiment of the disclosure.

FIG. 2 is a perspective view illustrating a sterilization robot according to an embodiment of the disclosure, viewed from one direction.

FIG. 3 is a perspective view illustrating a sterilization robot according to an embodiment of the disclosure, viewed from another direction.

FIG. 4 is a diagram illustrating sterilization ranges depending on arrangements of sterilization lamps in a conventional sterilization robot and a sterilization robot according to an embodiment of the disclosure.

FIG. 5 is a flowchart illustrating a method of controlling a sterilization robot according to an embodiment of the disclosure.

FIGS. 6 to 8 are diagrams illustrating traveling paths of a sterilization robot according to an embodiment of the disclosure.

FIG. 9 is a diagram illustrating movements of sterilization modules in a sterilization robot according to an embodiment of the disclosure.

FIG. 10 is a diagram referred to for describing an intensive care method based on control of a sterilization module in a sterilization robot according to an embodiment of the disclosure.

FIGS. 11 and 12 are diagrams illustrating an actuator in a sterilization robot according to an embodiment of the disclosure.

### BEST MODE

Description will now be given in detail according to exemplary embodiments disclosed herein, with reference to the accompanying drawings. For the sake of brief description with reference to the drawings, the same or equivalent components may be provided with the same reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms first, second, etc. may be used herein to describe various elements, these elements should not be limited by these terms. These terms are generally only used to distinguish one element from another.

It will be understood that when an element is referred to as being “connected with” another element, the element may be directly connected with the other element or intervening elements may also be present. In contrast, when an element is referred to as being “directly connected with” another element, there are no intervening elements present.

A singular representation may include a plural representation unless it represents a definitely different meaning from the context.

Terms such as “include” or “has” are used herein and should be understood that they are intended to indicate an existence of several components, functions or steps, disclosed in the specification, and it is also understood that greater or fewer components, functions, or steps may likewise be utilized.

A robot is a machine device capable of automatically performing a certain task or operation. The robot may be controlled by an external control device or may be embedded in the control device. The robot may perform tasks that are difficult for humans to perform, such as repeatedly processing only a preset operation, lifting a heavy object, performing precise tasks or a hard task in extreme environments.

In order to perform such tasks, the robot includes a driver such as an actuator or a motor, so that the robot may perform various physical operations, such as moving a robot joint.

Industrial robots or medical robots having a specialized appearance for specific tasks due to problems such as high manufacturing costs and dexterity of robot manipulation were the first to be developed.

Whereas industrial and medical robots are configured to repeatedly perform the same operation in a designated place, mobile robots have recently been developed and introduced to the market. Robots for use in the aerospace industry may perform exploration tasks or the like on distant planets that are difficult for humans to directly go to, and such robots have a driving function.

In order to perform the driving function, the robot has a driver, wheel(s), a frame, a brake, a caster, a motor, etc. In order for the robot to recognize the presence or absence of surrounding obstacles and move while avoiding the surrounding obstacles, an evolved robot equipped with artificial intelligence has recently been developed.

Artificial intelligence refers to a technical field for researching artificial intelligence or a methodology for implementing the artificial intelligence. Machine learning refers to a technical field for defining various problems handled in the artificial intelligence field and for researching methodologies required for addressing such problems. Machine learning is also defined as an algorithm that improves performance of a certain task through continuous experience.

An artificial neural network (ANN) is a model used in machine learning, and may refer to an overall model having problem solving ability, which is composed of artificial neurons (nodes) that form a network by a combination of synapses. The artificial neural network (ANN) may be defined by a connection pattern between neurons of different layers, a learning process of updating model parameters, and an activation function of generating an output value.

The artificial neural network (ANN) may include an input layer and an output layer, and may optionally include one or more hidden layers. Each layer includes one or more neurons, and the artificial neural network (ANN) may include a synapse that interconnects neurons and other neurons.

In the artificial neural network (ANN), each neuron may output a function value of an activation function with respect to input signals received through synapses, weights, and deflection.

A model parameter may refer to a parameter determined through learning, and may include the weight for synapse connection and the deflection of neurons. In addition, the hyperparameter refers to a parameter that should be set before learning in a machine learning algorithm, and includes a learning rate, the number of repetitions, a mini-batch size, an initialization function, and the like.

The purpose of training the artificial neural network (ANN) may be seen as determining model parameters that minimize a loss function according to the purpose of the robot or the field of use of the robot. The loss function may be used as an index for determining an optimal model parameter in a learning process of the artificial neural network (ANN).

Machine learning may be classified into supervised learning, unsupervised learning, and reinforcement learning according to learning methods.

Supervised learning refers to a method for training the artificial neural network (ANN) in a state where a label for learned data is given. Here, the label may refer to a correct answer (or a resultant value) that should be inferred by the artificial neural network (ANN) when the learned data is input to the artificial neural network (ANN). Unsupervised learning may refer to a method for training the artificial neural network (ANN) in a state where a label for learned data is not given. Reinforcement learning may refer to a learning method in which an agent defined in the certain environment learns to select an action or sequence of actions that may maximize cumulative compensation in each state.

Among artificial neural networks, machine learning implemented as a deep neural network (DNN) including a plurality of hidden layers is also referred to as deep learning, and deep learning is a part of machine learning. Hereinafter, machine learning is used in a sense including deep learning.

Artificial intelligence (AI) technology is applied to the robot, so that the robot may be implemented as a guide robot, a transportation robot, a cleaning robot, a wearable robot, an entertainment robot, a pet robot, and an unmanned aerial robot, etc.

The robot may include a robot control module for controlling operation thereof, and the robot control module may refer to a software module or a chip implemented in hardware.

By means of sensor information obtained from various types of sensors, the robot may acquire state information of the robot, may detect (recognize) the surrounding environment and the object, may generate map data, may determine a travel route and a travel plan, may determine a response to user interaction, or may determine a necessary operation.

The robot may perform the above-described operations using a learning model composed of at least one artificial neural network (ANN). For example, the robot may recognize the surrounding environment and object using a learning model, and may determine a necessary operation using the recognized surrounding environment information or object information. Here, the learning model may be directly learned from the robot or learned from an external device such as an AI server.

In this case, whereas the robot may perform a necessary operation by directly generating a result using the learning model, the robot may also perform an operation by transmitting sensor information to an external device such as an AI server and receiving the resultant information generated thereby.

The robot may perform autonomous driving through artificial intelligence. Autonomous driving refers to a technique in which a movable object such as a robot may autonomously determine an optimal path by itself and may move while avoiding collision with an obstacle. The autonomous driving technique currently being applied may include a technique in which the movable object (e.g., a robot) may travel while maintaining a current driving lane, a technique in which the movable object may travel while automatically adjusting a driving speed such as adaptive cruise control, a technique in which the movable object may automatically travel along a predetermined route, and a driving technique in which, after a destination is decided, a route to the destination is automatically set.

In order to perform autonomous driving, the movable object such as the robot may include a large number of sensors to recognize data of the surrounding situation. For example, the sensors may include a proximity sensor, an illumination sensor, an acceleration sensor, a magnetic sensor, a gyro sensor, an inertial sensor, an RGB sensor, an infrared (IR) sensor, a fingerprint recognition sensor, an ultrasonic sensor, an optical sensor, a microphone, a lidar, a radar, and the like.

The robot may perform autonomous driving not only based on information collected by sensors, but also based on image information collected by an RGBC camera and an infrared (IR) camera and sound information collected through a microphone. In addition, the robot may travel based on information received through a user input unit. Map data, location information, and information about peripheral situations may be collected through a wireless communication unit. The collected information is requisite for autonomous driving.

Map data may include object identification information for various objects disposed in a space where the robot moves. For example, the map data may include object identification information for fixed objects such as a wall and a door, and other object identification information for movable objects such as a flowerpot and a desk. In addition, the object identification information may include a name, a type, a distance, a location, etc.

Therefore, the robot may essentially include sensors, various input units, a wireless communication unit, and the like to collect data that may be learned by artificial intelligence, and may perform optimal operations by synthesizing various types of information. The learning processor for performing artificial intelligence may perform learning by being mounted in a controller embedded in the robot, may transmit the collected information to a server, may perform learning through the server, and may retransmit the learned result to the robot, so that the robot may perform autonomous driving based on the learned result.

A robot equipped with artificial intelligence may collect the surrounding information even in a new place to implement the entire map, and a large amount of information about a place of the major activity zone may be accumulated, so that the robot may perform more accurate autonomous driving.

The robot may include a touchscreen or a button to receive a user input, and may receive a command by recognizing a user's voice. In order to convert a voice input signal into a character string, the processor may obtain information about the intention corresponding to the user input using at least one of a speech to text (STT) engine for converting a voice input into a character string and a natural language processing (NLP) engine for obtaining information about the intention of natural language.

In this case, at least one of the STT engine and the NLP engine may include an artificial neural network (ANN) trained by a machine learning algorithm. In addition, at least one of the STT engine and the NLP engine may be trained by the learning processor, may be trained by the learning processor of the AI server, or may be trained by distributed processing of the trained results.

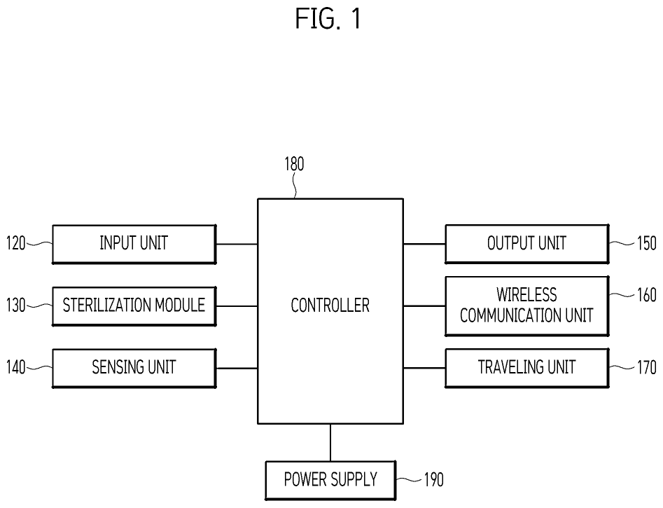

FIG. 1 is a block diagram illustrating the configuration of a sterilization robot 100 according to an embodiment of the disclosure.

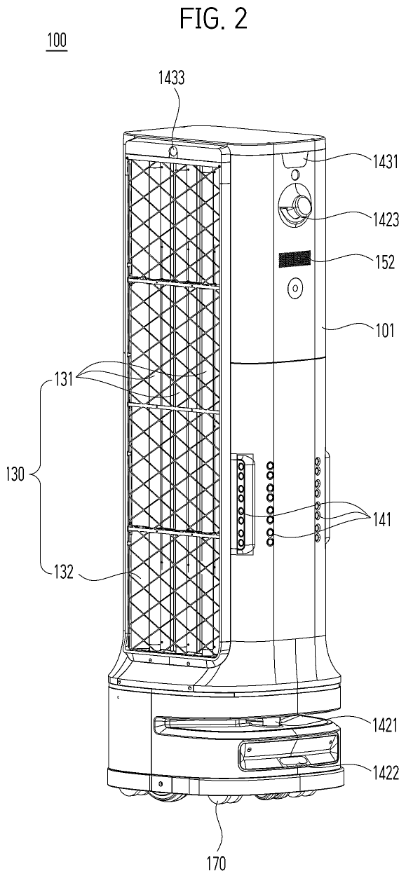

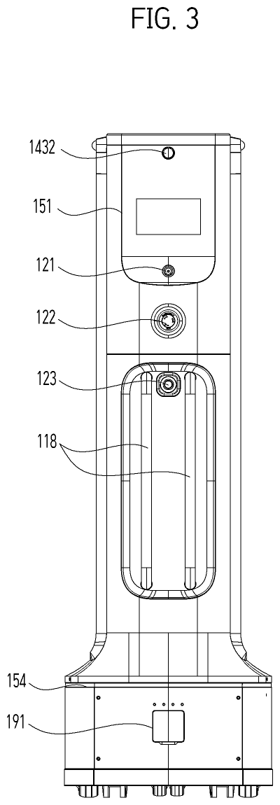

FIG. 2 is a front perspective view illustrating the sterilization robot 100 according to an embodiment of the disclosure, and FIG. 3 is a rear perspective view illustrating the sterilization robot 100 according to an embodiment of the disclosure.

As illustrated in FIGS. 2 and 3, the sterilization robot 100 of the disclosure may include an upright body 101 that extends in a vertical direction and sterilization modules 130 on both sides of the body 101.

Since the sterilization robot 100 aims to disinfect a user's hands and a location where the user's droplets reach, the body 101 may be configured to have a height of about 160 cm in consideration of the height of the user's mouth. The body 101 may be configured to have a height less than the height of an adult, as it may be difficult for the user to operate and difficult to stabilize if it is too tall.

The sterilization robot 100 of the disclosure may include a sterilization module 130 having a sterilization lamp 131 providing ultraviolet (UV) light, and a traveling unit 170 for moving the sterilization robot 100.

For stable traveling of the traveling unit 170, the sterilization robot 100 may include a sensing unit 140 for sensing the surroundings, an input unit 120 for inputting a control command, and an output unit 150 for providing information such as the state of the sterilization robot 100.

Further, the sterilization robot 100 may include a controller 180 for controlling the above components, a wireless communication unit 160 for connecting to an external server or terminal to receive map information or control information, and a power supply including a battery.

Some of the components illustrated in FIG. 1 are not essential to implement the sterilization robot 100, and the sterilization robot 100 described herein may have more or fewer components than those described above.

The sterilization module 130 may include the sterilization lamp 131 that irradiates UV light, and further include a reflector 132 on the rear surface of the sterilization lamp 131 to reflect light irradiated from the sterilization lamp 131 back to an object.

As illustrated in FIGS. 2 and 3, a pair of sterilization modules 130 of the disclosure may be provided on the left and right sides of the body 101. The sterilization robot 100 performs sterilization while moving, and there should be no obstacles on a traveling path of the sterilization robot 100, so that there are no objects to be sterilized in front of and behind the sterilization robot 100 in a traveling direction.

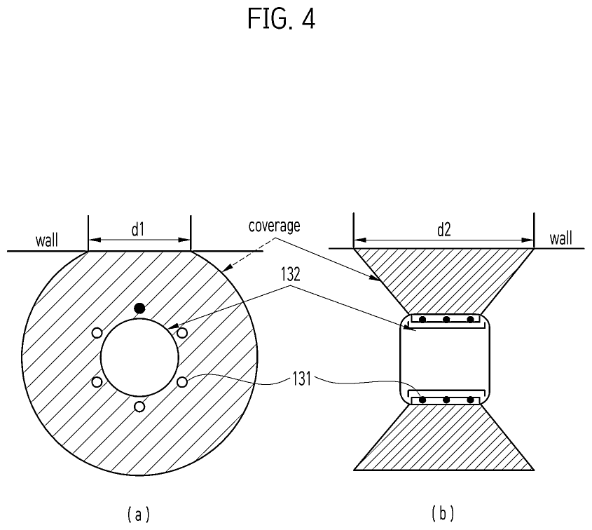

Therefore, when UV light is irradiated laterally in the traveling direction, sterilization may be performed more efficiently. FIG. 4 is a diagram illustrating sterilization ranges depending on arrangements of sterilization lamps 131 in a conventional sterilization robot 100 and the sterilization robot 100 according to an embodiment of the disclosure. The sterilization lamps 131 are arranged in a 360-degree direction in FIG. 4(a), and the sterilization lamps 131 are arranged laterally in a traveling direction in FIG. 4(b).

The sterilization effect of UV light irradiated from the sterilization lamps 131 decreases as a distance increases, and an effective sterilization distance may be about 1 m. Therefore, a sterilization range D2 of the sterilization lamps 131 arranged side by side in a lateral direction as illustrated in FIG. 4(b) may be wider than a sterilization range DI of the sterilization lamps 31 arranged along the periphery of the body 101 of the sterilization robot 100.

The sterilization robot 100 may move, spaced apart from objects by a smaller distance than the effective sterilization distance to increase sterilization power, and when the sterilization modules 130 are retracted into, withdrawn from, or tilted to the body 101, the positions and directions of the sterilization modules 130 may be adjusted so that the sterilization modules 130 are as close to the objects as possible. A sterilization module 130 may also be segmented in the vertical direction and driven separately, so that the modules may be driven separately when sterilizing objects of different sizes depending on their heights.

The sterilization robot 100 may further include a protective cover that covers the front surfaces of the sterilization lamps 131 (a side surface of the sterilization robot 100) to protect the sterilization lamps 131. The sterilization lamps 131 may have a defined lifetime and be replaced by removing the protective cover.

The traveling unit 170 may include a motor 1351 and a wheel for moving the body 101 of the sterilization robot 100, and may be located in a lower part of the body 101. The traveling unit 170 may include a plurality of wheels for stable movement, and the wheels may include a wheel connected to the motor to provide a main driving force and a caster to adjust a direction.

As there are more wheels, movement is more stable. The sterilization robot 100 may be equipped with multiple small-sized wheels, because stable driving and a wheel structure that may cope with obstacles are more important than fast driving. Further, since the wheels support the weight of the entire body 101, they have a certain rigidity.

The sensing unit 140 is a device for sensing information within the sterilization robot 100 or environmental information around the sterilization robot 100. Particularly, since the sterilization robot 100 includes a traveling function, a proximity sensor 141 for recognizing an obstacle around the sterilization robot 100 may play an important role to enable stable movement.

Upon detection of an event corresponding to a specific condition, the sensing unit 140 may generate a sensing signal, and the controller 180 may control the traveling unit 170 or the sterilization modules 130 based on the sensing signal.

The sterilization robot 100 according to an embodiment of the disclosure may include, as the sensing unit 140, the proximity sensor 141, lidars 1421, 1422, and 1423, and motion sensors 1431, 1432, 1433 on the body 101.

As illustrated in FIG. 2, the sterilization robot 100 may be provided with a UV sensor to detect a nearby object in the traveling direction. UV sensors may be located on the front and side surfaces of the body 101.

Because the sterilization modules 130 occupy most of the areas of the side surfaces of the body 101, the UV sensors may be disposed at positions close to the front surface to avoid the sterilization modules 130, and a plurality of UV sensors may be provided in the vertical direction to detect an obstacle of an upwardly protruding shape.

As illustrated in FIG. 2, a plurality of lidars 1421, 1422, and 1423 may be provided. A first lidar 1421 located in the lower part may be positioned in a horizontally elongated recess and detect an obstacle in a horizontally wide area. The recess may extend to the middles of the side surfaces to allow the lidar to cover a range of 180 degrees or more.

A second lidar 1422 may be disposed to be upwardly inclined to detect an object above it, and a third lidar 1423 may be disposed downwardly inclined to detect an object below it.

A lidar is a device that emits laser pulses and receives the light reflected back from nearby objects to create a precise picture of the surroundings. The lidar is similar to the radar in principle, except for used electromagnetic waves, and thus the lidar and the radar use different technologies and are used in different applications.

A laser uses light in a wavelength between 600 and 1000 nm, which may damage human vision. The lidar uses a longer wavelength and is used to measure the distance, speed and direction of movement of a target object, and temperature, analyze atmospheric substances in the environment, and measure the concentrations of the substances.

The controller 180 may use the lidars 1421, 1422, and 1423 to recognize objects in the surroundings and construct a map based on information sensed by the lidars 1421, 1422, and 1423.

Motion sensors may also be disposed on a front surface 1431, a rear surface 1432, and side surfaces 1433 of the body 101, respectively. These are sensors that detect moving objects, and UV light may have an effect on living organisms when directly irradiated.

Direct irradiation of UV light from the sterilization lamps into the eyes may cause problems such as blindness, and destroy skin cells. Accordingly, when a human is near the sterilization robot 100, a sterilization operation may be stopped (sterilization lamps OFF).

Particularly, pets or babies may approach the sterilization robot 100 curiously. Therefore, the sterilization robot 100 may turn off the sterilization modules 130 or stop movement by detecting the approach of an unexpected creature.

The output unit 150, which is for generating an output associated with visual, auditory, or tactile sensations, may include at least one of a display unit 151, a speaker 152, or an optical output unit 154.

The display unit 151 may be layered with or integrally formed with a touch sensor, thereby implementing a touch screen. The touch screen may function as the user input unit 120 for providing an input interface between the sterilization robot 100 and the user, and also provide an output interface.

The optical output unit 150 may use a light source of a variable color to facilitate recognition of the state of the sterilization robot 100, and may include an LED lamp 154 positioned on the rear surface, as illustrated in FIG. 3. A state such as sterilizing, charging, fully charged, low battery, and so on may be displayed through the optical output unit 150.

The input unit 120 may include a camera or video input unit 120 for inputting a video signal, a microphone or audio input unit 120 for inputting an audio signal, and a user input unit 120 (e.g., a touch key, a mechanical key, and so on) for receiving information from a user. Audio data or image data collected from the input unit 120 may be analyzed and processed into a control command from the user.

In addition to a user input method using the display unit 151, a physical button may be exposed to the outside for use in an emergency or power-off state, and located on the rear surface for the purpose of outward appearance and for operation while viewing the display located on the rear surface, as illustrated in FIG. 3.

The user input unit may include a power button 121, an emergency stop button 122, and a manual operation button 123. The power button 121 is a button to activate the sterilization robot 100. The emergency stop button 122, which is a button for the user to force the sterilization robot 100 to stop operation in an emergency situation, may stop only movement or turn off even the sterilization modules 130.

The manual operation button 123 is a device for unlocking the traveling unit 170 which has been locked, for human transportation of the sterilization robot 100.

When the traveling unit 170 is not locked, the sterilization robot 100 may easily move or fall over due to an external impact after the sterilization robot 100 completes its operation. On the contrary, when the sterilization robot 100 is pushed while locked, a locking device or wheels may be broken, and the sterilization robot 100 is heavy to lift and move.

Therefore, when the user pushes the sterilization robot 100 while pressing the manual operation button 123, the sterilization robot 100 moves, and the sterilization robot 100 may be provided with a movement handle 118 next to the manual operation button 123 so that the user may easily move the sterilization robot 100 while pressing the manual operation button 123.

The wireless communication unit 160 may include one or more modules that enable wireless communication between the sterilization robot 100 and a wireless communication system, between the sterilization robot 100 and an external terminal, or between the sterilization robot 100 and an external server. The wireless communication unit 160 may include at least one of a mobile communication module, a short-range communication module, or a location information module.

The mobile communication module transmits and receives wireless signals to and from at least one of a base station, an external terminal, or a server over a mobile communication network established according to a technology standard or communication scheme for mobile communication (e.g., long term evolution (LTE), long term evolution-advanced (LTE-A), or the like).

The short-range communication module, which is for short-range communication, may support short-range communication using at least one of Bluetooth™, radio frequency identification (RFID), infrared data association (IrDA), ZigBee, near field communication (NFC), or wireless-fidelity (Wi-Fi).

As such, the short-range communication module may support wireless communication between the sterilization robot 100 and a wireless communication system, between the sterilization robot 100 and another sterilization robot 100, or between the sterilization robot 100 and a network in which another terminal is located, through a wireless area network. The wireless area network may be a wireless personal area network.

The short-range communication module may exchange data with another terminal or another sterilization robot 100, and may be used to detect a device such as a communicable terminal or robot in the vicinity. Data may be transmitted and received by connecting to the detected device, and the sterilization robot 100 may be controlled remotely when connected to the user's terminal or a controller using the short-range communication module.

The location information module is a module for acquiring the location (or current location) of the sterilization robot 100, and its representative example is a global positioning system (GPS) module or a Wi-Fi module. For example, the sterilization robot 100 may use the GPS module to obtain the location of the sterilization robot 100 using signals from GPS satellites. In another example, the sterilization robot 100 may use a Wi-Fi module to obtain the location of the sterilization robot 100 based on information from a wireless access point (WAP) that transmits or receives wireless signals to or from the Wi-Fi module.

The controller 180 may set a traveling path using ambient information detected by the input unit 120 or the sensing unit 140, and receive a map or a user command through the wireless communication unit 160. The controller 180 may control the traveling unit 170 to move the sterilization robot 100 along the traveling path, and selectively turn the sterilization modules 130 on/off to perform optimal sterilization while saving energy.

Further, the controller 180 may control the output unit 150 to provide the user with information about the state of the sterilization robot 100, sterilization information, and so on.

The power supply 190 may supply power to the sterilization lamps 131 and the traveling unit 170, and may be equipped with a battery, for free movement. The weighted battery may be located in the lower part to enable stable traveling, and a charging terminal for charging the battery may be provided on the rear surface. A charging terminal cover 191 may be further included to protect the charging terminal and prevent it from being exposed to the outside.

The sterilization robot 100 of the disclosure sterilizes furniture, appliances, and other parts that may be touched or contaminated by human hands, while moving around indoors. The sterilization robot 100 may reduce a sterilization time and minimize power consumption by sterilizing objects to be sterilized such as furniture, home appliances, and so on, while traveling at a predetermined distance (sterilization distance) from the objects, and sterilizing for a shorter time or not sterilizing objects that are not directly touched by the user and are not easily contaminated.

For example, frequently touched parts such as door handles or beds may be highly contaminated and thus require a longer sterilization time or an increased intensity of the sterilization lamps 131. Since walls are not directly touched by the user, sterilization of a corresponding area may take less time than sterilization of highly contaminated furniture or appliances, or may be omitted.

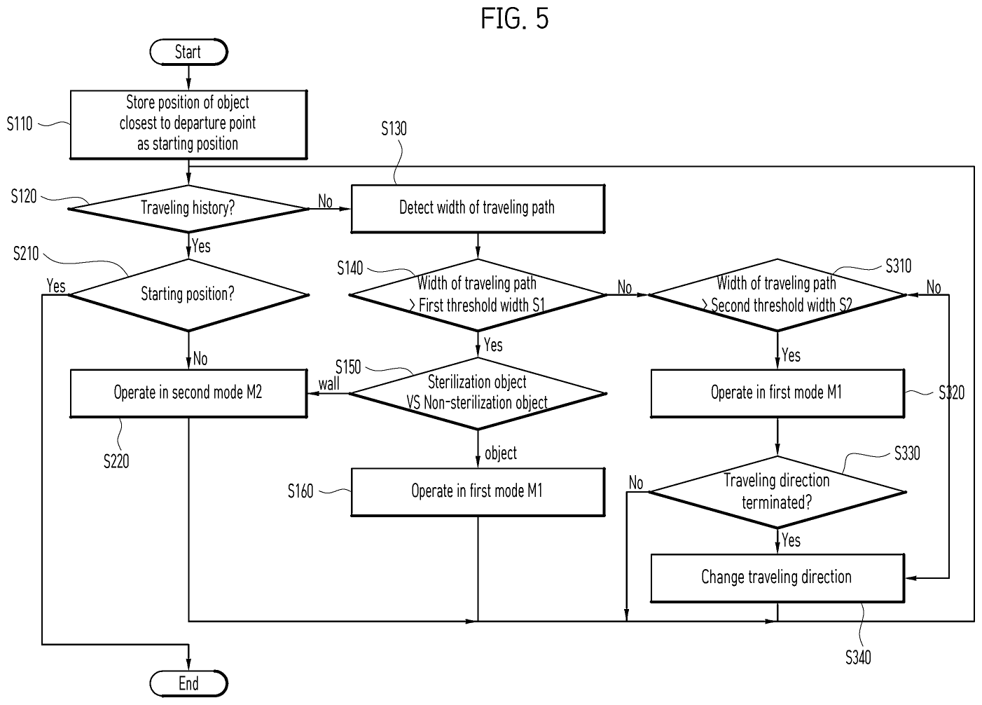

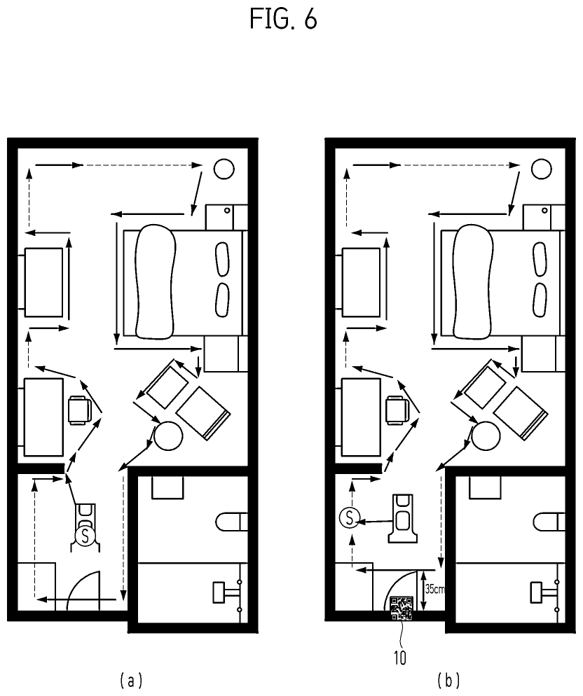

FIG. 5 is a flowchart illustrating a method of controlling the sterilization robot 100 according to an embodiment of the disclosure, and FIGS. 6 to 8 are diagrams illustrating traveling paths of the sterilization robot 100 according to an embodiment of the disclosure.

When the sterilization robot 100 starts operation, an ending point may be marked by saving a departure point for traveling as a starting position S (S110).

However, since the sterilization robot 100 moves by calculating a traveling path by detecting objects such as walls, furniture, home appliances, and so on as described above, a problem arises that the sterilization robot 100 is not able to return to the saved starting point S when a location spaced apart from a wall or a sterilization object by a sterilization distance or larger is saved as the starting point S, as illustrated in FIG. 6(a).

Therefore, when the sterilization robot 100 starts operation, the starting point S may be saved after the sterilization robot 100 detects an object such as a wall, furniture, or an appliance and moves to the vicinity of the object, as illustrated in FIG. 6(b).

When an object in a traveling direction is detected and there is no previous travel history for the object (i.e., the object has never been sterilized) (S120), a traveling path may be set. The traveling path may be set based on an existing map, if available, or may be generated in real time using a sensor such as the proximity sensor 141 or a lidar.

To set the traveling path, the controller 180 may detect a travelable width using the sensing unit 140 to set the traveling path (S130). The controller 180 may calculate the travelable width before setting the traveling path to set the traveling path in consideration of the width of the sterilization robot 100 and the sterilization distance of the sterilization robot 100.

When the travelable width is equal to or greater than a first threshold width (S140), the controller 180 may set a first traveling path to travel adjacent to objects, and operate the sterilization robot 100 in a first mode (S160). The first threshold width may be set based on the width w of the sterilization robot 100 and an effective sterilization distance e.

For example, the sum of the width w of the sterilization robot 100 and a distance twice the effective sterilization distance e may be set as the first threshold width, and when the first threshold width is exceeded, the sterilization robot 100 may be travel close to the objects. When the width of the sterilization robot 100 is 50 cm and the effective sterilization distance is 1 m, the first threshold width is 2.5 m, and it is determined whether the width of a travelable space ahead is 2.5 m or more.

When the width of the travelable space ahead is greater than 2.5 m, the controller 180 may set the first traveling path (indicated by a solid line in FIG. 6) to move along objects on one side.

Before setting the first traveling path, it may be determined whether an object is a sterilization object or a non-sterilization object that does not require sterilization (S150), and when the object is a sterilization object that requires sterilization, the first traveling path may be set and the sterilization robot 100 may be operated in the first mode and perform sterilization (S160).

The first mode refers to a method of traveling while sterilizing a sterilization object located on one side, wherein a traveling path in which the sterilization robot 100 travels at a first distance from a sterilization object may be set.

The traveling path may be set such that the sterilization robot 100 travels at a predetermined distance (the first distance) from surrounding objects, and in this embodiment, the sterilization distance is set to 10 cm. The sterilization distance may be set to 10 cm or more for a wider range of sterilization, and the sterilization distance may be freely set within the effective sterilization distance.

Further, since sterilization objects located on one side are sterilized, the sterilization robot 100 may travel with a sterilization module 130 facing the sterilization object turned on and a sterilization module 130 located on the opposite side turned off.

When there is an object to be sterilized, the sterilization robot 100 may travel at a first speed with the sterilization module 130 turned on. In the first mode, the sterilization robot 100 may travel at the first speed, and the controller 180 may determine the first speed to sufficiently irradiate UV light in consideration of the intensity of the sterilization lamps 131 of the sterilization module 130 and the sterilization distance. In this embodiment, the sterilization robot 100 may travel at a speed of 3 to 5 cm/s in the first mode. Further, when the sterilization object on the traveling path is a door, the sterilization robot 100 may travel at a second distance greater than the afore-described first distance, as illustrated in FIG. 6(b).

The second distance is for securing a space allowing the door to be opened in consideration that when the sterilization robot 100 stops in front of the door due to an error and discontinues its operation, entry is not possible.

For example, at a distance of about 35 cm, sterilization is possible and a space may be secured, in which the door is opened for a person to enter.

Although the location of the door may be identified by the sterilization robot 100 through a sensor, it may be difficult to distinguish the door from a wall. Accordingly, the door may be provided with a marker 10, such as a QR code, so that the sterilization robot 100 may identify whether it is a door.

When the object is a non-sterilization object that does not require sterilization, such as a wall, the sterilization robot 100 may be operated in a second mode (S150 and S220). Since the second mode is for an area that does not require sterilization, the sterilization robot 100 may turn off the sterilization lamps 131 and travel at a second speed greater than in the first mode.

Turning off the sterilization lamps 131 in the second mode in which sterilization is not required may prevent discoloration caused by UV irradiation, save battery power of the sterilization robot 100, and shorten the sterilization time.

The second speed is greater than the afore-described first speed, and may be set to a speed that is not too large since the sterilization robot 100 moves indoors. For example, the second speed may be set to 50 to 70 cm/s.

A second traveling path for the second mode may be set at a certain distance from objects as in the first traveling path. However, since sterilization is not performed in the second traveling path, the distance from the objects is not important as in the first traveling path, and thus a shortest path for fast passing by non-sterilization objects may be set as the second traveling path.

The second mode may be optionally implemented and performed in the same manner as the first mode, without determining whether an object is to be sterilized. Alternatively, in the second mode, the controller 180 may operate the sterilization robot 100 at the same speed as in the first mode, with only the sterilization lamps 131 turned off, or may control the traveling unit 170 differently from in the first mode, such that the sterilization robot 100 travels faster than the first speed of the first mode, with the sterilization lamps 131 turned on.

When the width of a traveling space ahead is less than or equal to the first threshold width and equal to or greater than a second threshold width, a third traveling path may be set and the sterilization robot 100 may be operated in the third mode (S310 and S320).

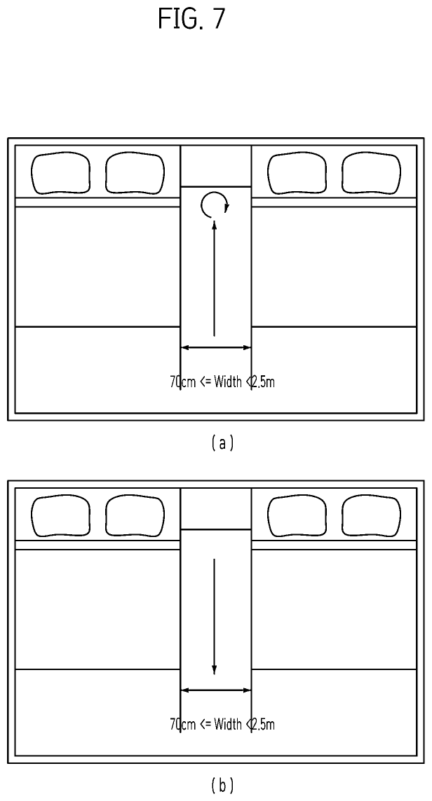

FIG. 7 is a diagram illustrating a method of traveling in the third traveling path in the third mode. FIG. 7(a) is a diagram illustrating entering along the third traveling path, and FIG. 7(b) is a diagram illustrating turning around and traveling back along the third traveling path.

The first threshold width means a maximum width allowing simultaneous bilateral sterilization, and the second threshold width means a minimum width allowing the sterilization robot 100 to enter. That is, when a space has a width equal to or less than the second threshold width, the sterilization robot 100 is not capable of entering or returning even if it enters, and a traveling path may be omitted for a space having a width equal to or less than the second threshold width.

In an embodiment where the width of the sterilization robot 100 is 50 cm, the second threshold width may be set to 70 cm, and when the afore-described first threshold width is 250 cm, the third traveling path may be set for a space having a width greater than 70 cm and equal to or less than 250 m.

The third traveling path is for an area where objects on both sides may be sterilized simultaneously, and the controller 180 may set the middle of the travelable width as the third traveling path. In the third mode, the sterilization modules 130 on both sides may be turned on to sterilize both sides simultaneously.

In this case, since the distance to sterilization objects is greater than in the first mode described above, the sterilization robot 100 may be operated at a third speed less than the first speed or with an increased intensity of the sterilization lamps 131.

When a path is blocked during traveling and further traveling is not possible, the traveling direction may be changed (S330 and S340). Herein, the forward and backward directions of the sterilization robot 100 may be switched using the traveling unit 170, or the sterilization robot 100 may travel in reverse along the third traveling path and exit the path by driving the traveling unit 170 reversely.

Herein, since the area has already been sterilized, the sterilization robot 100 may travel faster than the speed of the third mode and travel with the sterilization lamps 131 turned off. That is, the sterilization robot 100 may travel in the second mode described above.

When there is a traveling history for the traveling path set in the traveling direction (S120), the controller 180 may control the sterilization robot 100 to turn off the sterilization lamps 131 and move at a fast speed, determining that the traveling path has already been sterilized. In other words, the controller 180 may control the traveling unit 170 and the sterilization modules 130 to operate in the second mode described above (S220).

This may be understood as operation in the second mode even for traveling in reverse in the third traveling path described above, since there is a traveling history for the third traveling path.

However, when the stored starting position S is matched, it may be determined that the sterilization is completed and the operation may be terminated (S210).

When there is still a space requiring sterilization even though the sterilization robot 100 arrives at the starting position S, that is, when there is a space for which the controller 180 may set an additional traveling path, the controller 180 may set an additional traveling path without terminating sterilization and continue operating the sterilization robot 100.

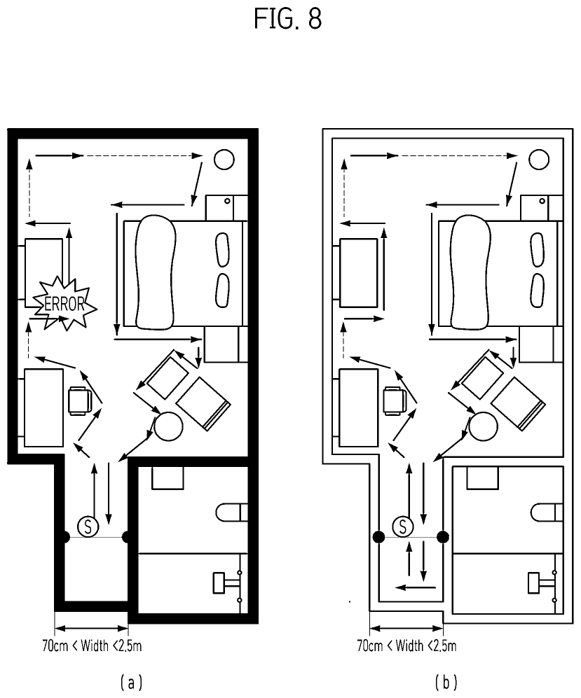

For example, when the width of a traveling space at the starting position S is for the third traveling path described above, the sterilization robot 100 moves along the center of the traveling space. Accordingly, although the sterilization robot 100 arrives at the starting position S, there remains a space that has not yet been sterilized, as illustrated in FIG. 8(a).

In other words, even if the sterilization robot 100 reaches the starting position S, a traveling path may be set in a direction that has no traveling history, and thus the sterilization robot 100 may continue the sterilization operation without terminating the traveling. In this case, the third traveling path may be set as illustrated in FIG. 7, and the sterilization robot 100 may travel and return along the third traveling path and stop at the starting point S (see FIG. 8(b)).

Upon occurrence of an error during traveling, the sterilization robot 100 may stop traveling and notify an operator of the error at a point where the error occurs. The error may be indicated visually through the display unit, audibly, or by transmitting an error notification to a terminal connected to the sterilization robot 100, so that the operator may be notified of the error.

Upon selection of Continue, the sterilization robot 100 may set a traveling path again and travel. In this case, the sterilization robot 100 may keep the previously stored starting point S and stop the operation when arriving at the previously stored starting point S, rather than re-save the error point as a starting point.

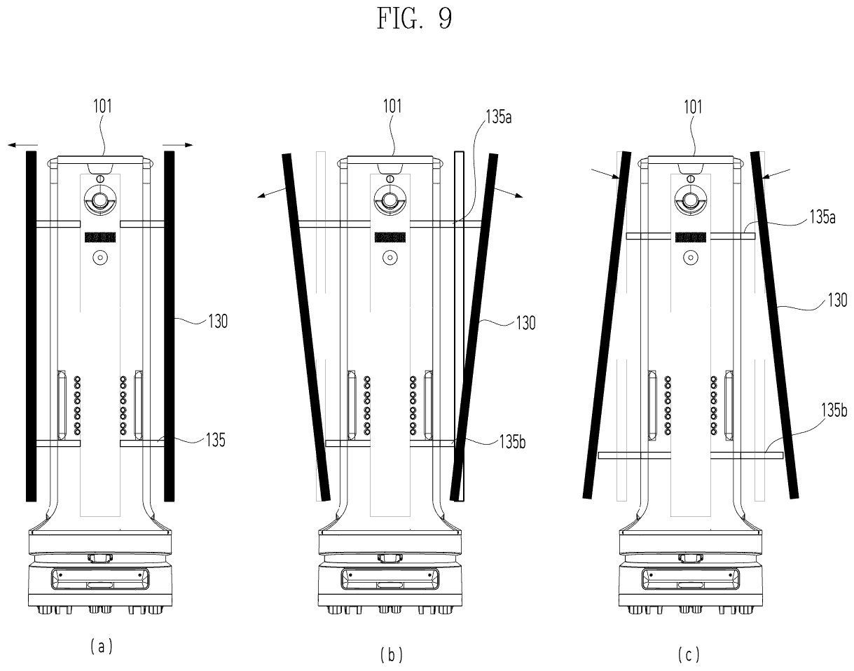

FIG. 9 is a diagram illustrating movements of the sterilization modules 130 in the sterilization robot 100 according to an embodiment of the disclosure. The sterilization modules 130 in the sterilization robot 100 of the disclosure may change its distances from the body 101 as illustrated in FIG. 9(a) or adjust their angles as illustrated in FIGS. 9(b) and 9(c).

The sterilization module 130 of the disclosure may further include an actuator 135 of a variable length on its rear surface. The actuator 135 is positioned between a frame and the sterilization modules 130 inside the body 101, and as the length of the actuator 135 changes, the sterilization modules 130 may be moved away from or closely contact the body 101.

The actuator 135 may include a plurality of actuators in upward and downward directions for each sterilization module 130, such that when the length of the actuator 135 is changed, the angles of the sterilization modules 130 may be changed, as illustrated in FIGS. 9(b) and 9(c). To change the angles of the sterilization modules 130, ball joints 1356 may be provided on ends of the actuators 135 and the sterilization modules 130.

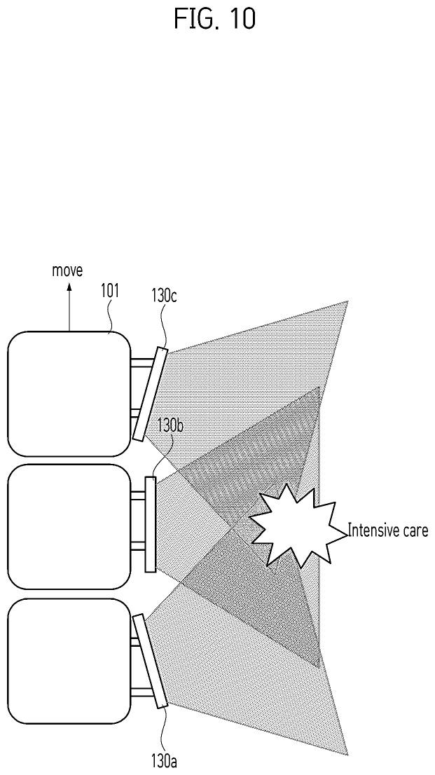

When a plurality of actuators 135 are provided in a horizontal direction, an angle in the horizontal direction may also be changed, as illustrated in FIG. 10. When sterilizing a low sterilization object such as a bed, the controller 180 may adjust the angles of the sterilization modules 130 by controlling the length of an upper actuator 135a to be greater than the length of a lower actuator 135b so that the upper actuator 135a faces downward, as illustrated in FIG. 9(b).

Alternatively, to sterilize a part located higher than the sterilization robot 100, the controller 180 may control the lengths of the upper and lower actuators 135a and 135b to be different so that the sterilization modules 130 face upward, as illustrated in FIG. 9(c).

FIG. 10 is a diagram illustrating an intensive care method by controlling the sterilization modules 130 in the sterilization robot 100 according to an embodiment of the disclosure. It is necessary to increase an sterilization intensity in areas that are intensively touched by human hands, such as door handles.

In other words, when sterilizing a sterilization object that needs to be sterilized in an intensive care mode, the controller 180 may increase the intensity of the sterilization lamps 131 or adjust a speed, and may adjust the angles of the sterilization modules 130 to increase the duration of UV irradiation to the sterilization object as illustrated in FIG. 10.

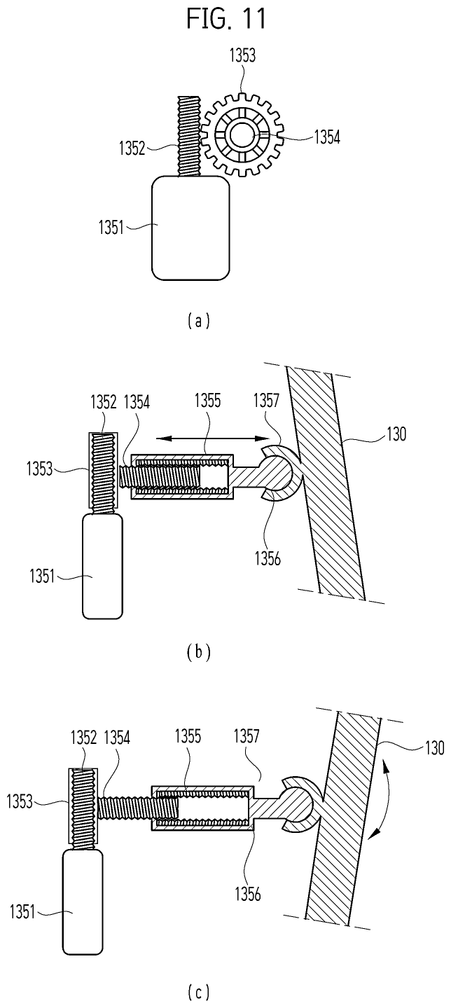

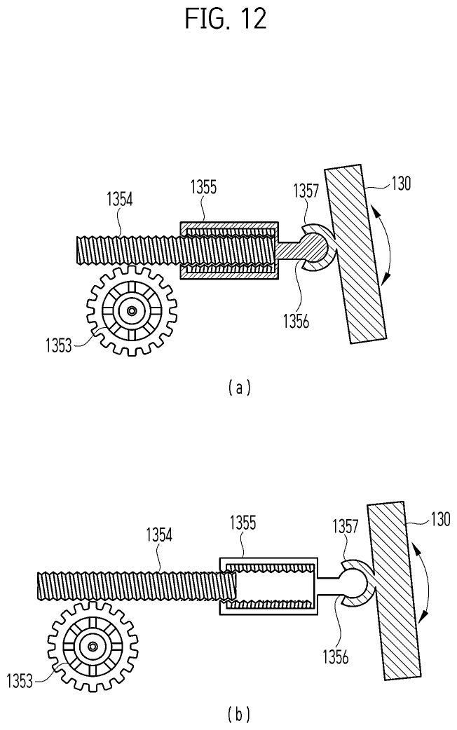

FIGS. 11 and 12 are diagrams illustrating the actuator 135 in the sterilization robot 100 according to an embodiment of the disclosure. As described above, the actuator 135 of the disclosure may include a motor 1351 that may change the length of the actuator 135 from the body 101, and a gear and a screw that receive rotational force from the motor 1351 and translate rotation to linear motion.

FIG. 11(a) is a diagram illustrating the actuator 135 viewed from a side of the sterilization robot 100, and FIGS. 11(b) and 11(c) are diagrams illustrating the actuator 135 viewed from the front of the sterilization robot 100, wherein aspects of changing the length of the actuator 135 are illustrated.

The actuator 135 may include a first screw 1354 that rotates by receiving rotational force from the motor 1351, and a drive block 1355 that includes a thread into which the first screw 1354 is inserted and that changes its position on the first screw 1354 when the first screw 1354 rotates. An end of the drive block 1355 may be coupled to the rear surface of a sterilization module 130 using a ball joint 1356, such that an angle between the end of the drive block 1355 and the sterilization module 130 may be freely changed.

When the positions of drive blocks 1355 of the plurality of actuators 135 are changed differently, the angles of the sterilization modules 130 may change. That is, the controller 180 may control the positions of the drive blocks 1355 through motors 1351, and consequently control the positions and angles of the sterilization modules 130.

The motor 1351 and the first screw 1354 may be directly connected, as illustrated in FIG. 12, or a rotation direction may be switched and rotational force may be transmitted to the first screw 1354 by using a second screw 1352 and a gear 1353 directly connected to the motor 1351 as illustrated in FIG. 11.

The embodiment of FIG. 12 may simplify the configuration by reducing the number of components. In the embodiment of FIG. 11, as the motor 1351 is disposed in the vertical direction, no force is exerted in a direction perpendicular to an extension direction of the second screw 1352 connected to the motor 1351.

Since load applied to the motor 1351 is small, the motor 1351 may rotate at a uniform speed, and it is more advantageous in terms of the stability of the motor 1351 to dispose the motor 1351 in the vertical direction as illustrated in FIG. 11.

In addition to the foregoing embodiment, the actuator 135 may also be implemented by using a rack-and-pinion gear that moves linearly by receiving rotational force from the motor 1351.

The sterilization robot 100 according to at least one embodiment of the disclosure may increase sterilization efficiency by controlling the traveling unit and the sterilization modules in an optimal manner.

Further, the sterilization robot 100 according to at least one embodiment of the disclosure may minimize discoloration caused by UV irradiation by avoiding sterilization of unnecessary parts and redundant sterilization.

Further, the sterilization robot 100 according to at least one embodiment of the disclosure may determine whether sterilization is necessary and, when sterilization is unnecessary, increase a traveling speed to quickly complete a sterilization operation.

Further, as the sterilization robot 100 includes a side surface without a sterilization lamp, the user may access and operate the sterilization robot 100 to cope with an emergency.

Further, UV light may be efficiently irradiated toward a sterilization object by adjusting the positions and angles of the sterilization modules 130.

An accurate traveling path may be set using sensors such as the lidars 1421, 1422, and 1423 that detect movement in various directions and the proximity sensor 141.

The above detailed description is not to be construed as limiting in any respect and should be considered exemplary. The scope of the disclosure is to be determined by a reasonable interpretation of the appended claims, and all changes within the equivalents of the disclosure are included in the scope of the disclosure.

1. A sterilization robot comprising:
a case;
a traveling unit located in a lower part of the case and providing a traveling function;
a sterilization module located on a side surface of the case;
a sensing unit detecting a nearby object; and
a controller setting a traveling path based on object information detected by the sensing unit and controlling the traveling unit to move along the traveling path.

2. The sterilization robot of claim 1, wherein the controller determines a sterilization object among objects detected by the sensing unit, and sets the traveling path to pass by the sterilization object while maintaining a sterilization distance from the sterilization object.

3. The sterilization robot of claim 2, wherein when a movement-direction width of the sterilization robot is equal to or greater than a first threshold width, the controller sets a first travel path to maintain a first distance from the sterilization object and operates the sterilization robot in a first mode.

4. The sterilization robot of claim 3, wherein a pair of sterilization modules are located on both sides in a direction perpendicular to a traveling direction of the traveling unit, and
wherein the controller turns on a sterilization module facing the sterilization object and turns off a sterilization module located opposite thereto, in the first mode.

5. The sterilization robot of claim 4, wherein when there is no sterilization object among the detected objects, the controller sets a second traveling path for movement along a wall and operates the sterilization robot in a second mode.

6. The sterilization robot of claim 5, wherein the traveling unit moves at a first speed in the first mode and at a second speed in the second mode.

7. The sterilization robot of claim 5, wherein the controller turns off the sterilization modules in the second mode.

8. The sterilization robot of claim 3, wherein when the movement-direction width of the sterilization robot has a value between a second threshold width and the first threshold width, the controller sets a third traveling path running along a middle of the movement-direction width and operates the sterilization robot in a third mode.

9. The sterilization robot of claim 8, wherein a pair of sterilization modules are located on both sides in a direction perpendicular to a traveling direction of the traveling unit, and
wherein the controller turns on the pair of sterilization modules in the third mode, and controls the traveling unit to move at a third speed.

10. The sterilization robot of claim 8, wherein when the third traveling path ends at a dead end, the controller controls the traveling unit to reverse a traveling direction turn around, and move back along the third traveling path.

11. The sterilization robot of claim 3, wherein when the sterilization object is a door, the first traveling path is set to be spaced from the door by a second distance greater than the first distance.

12. The sterilization robot of claim 1, wherein when the set traveling path overlaps with a previously traveled traveling path, the controller controls the traveling unit to move at a speed greater than a first speed used to move along the previously traveled traveling path.

13. The sterilization robot of claim 1, wherein the controller stores a traveling starting point by moving to an object at a location closest to a departure point, and upon arrival at the traveling starting point, turns off the traveling unit and the sterilization module.

14. The sterilization robot of claim 13, wherein when an unsterilized area remains upon arrival at the traveling starting point, the controller controls the traveling unit and the sterilization module by setting a traveling path that does not overlap with a previously traveled traveling path.

15. The sterilization robot of claim 1, wherein the sterilization module includes:
a sterilization lamp emitting ultraviolet light; and
a reflector reflecting the ultraviolet light on a rear surface of the sterilization lamp.

16. The sterilization robot of claim 15, wherein the sterilization module includes an actuator protruding from the case or changing an angle.

17. The sterilization robot of claim 16, wherein the actuator includes a plurality of actuators on a rear surface of the sterilization module, and
wherein the controller controls a position and a direction of the sterilization module by controlling a length change of the actuators.

18. The sterilization robot of claim 17, wherein when the sterilization object is highly contaminated, the controller changes the direction of the sterilization module during traveling of the sterilization robot by controlling the actuator to direct the sterilization module toward the sterilization object.
